(19) United States
(12) Patent Application Publication
(10) Pub. No.: US 2021/0035667 A1
(43) Pub. Date: Feb. 4, 2021

(54) SERVER, SYSTEM, AND SERVICE PROVISION METHOD

(71) Applicants: TOYOTA JIDOSHA KABUSHIKI KAISHA, Toyota-shi (JP); DENSO CORPORATION, Kariya-city (JP)

(72) Inventors: Yukari SUZUKI, Setagaya-ku (JP); Naoki YAMADA, Toyota-shi (JP); Toshihiro ARAI, Nagoya-shi (JP); Kuniaki JINNAI, Nagoya-shi (JP); Keiichi UNO, Chita-gun (JP)

(73) Assignees: TOYOTA JIDOSHA KABUSHIKI KAISHA, Toyota-shi (JP); DENSO CORPORATION, Kariya-city (JP)

(21) Appl. No.: 16/943,021

(22) Filed: Jul. 30, 2020

(30) Foreign Application Priority Data
Jul. 30, 2019 (JP) 2019-140171

(51) Int. Cl. G16H 10/60; G16H 40/67; G16H 40/63; G16H 50/20; G16H 20/30

(52) U.S. Cl. CPC G16H 10/60; G16H 40/67; G16H 20/30; G16H 50/20; G16H 40/63

(57) ABSTRACT

Provided is a system comprising a server configured to provide a service related to health of a user, and a vehicle configured to provide the service. The server decides a service to be provided to the user based on health checkup information of the user, and generates a dispatch schedule of the vehicle configured to provide the service. The vehicle is loaded with equipment or goods for providing the service, and is driven based on the dispatch schedule.

### CROSS-REFERENCES TO RELATED APPLICATIONS

The present application claims the benefit of Japanese Patent Application No. 2019140171, filed in the Japan Patent Office on Jul. 30, 2019, which is incorporated by reference herein in its entirety.

### TECHNICAL FIELD

The present disclosure relates to a server, a system, and a service provision method, and more particularly, to a server, a system, and a service provision method for providing a service related to health of a user.

### BACKGROUND

Recently, various services have been implemented using a means of transportation such as an automobile. Such services may include, as a type of nursing service business, a shuttle service business (for example, a day care service) that provides, along with a nursing service at a business facility, a transportation business to transfer customers in cyclic order by a nursing vehicle capable of carrying such equipment as wheelchairs and stretchers.

Furthermore, PTL 1 proposes providing, to a round welcoming and sending service enterprise using a nursing vehicle, a service of generating a dispatch/patrol plan by referencing nursing vehicle specifications and customer information, and also proposes providing, to a vehicle company, a nursing vehicle consulting for proposing a vehicle according to the characteristics of a round welcoming and sending service enterprise as a customer or providing marketing data on vehicle development.

### CITATION LIST

### Patent Literature

PTL 1: JP200556132A

### SUMMARY

A variety of services have been provided to directly or indirectly support people in need of nursing care; however, more services are still needed for providing, in the aim of reducing the medical expenses for a society as a whole, health maintenance and management for apparently healthy people.

For example, “health checkups” are indispensable for the prevention and early detection of diseases. However, most healthy people, who would enjoy no direct merit or incentive for undergoing checkups, do not undergo health checkups. Moreover, some people do not have enough knowledge about how to effectively utilize the results of the health checkup they had.

Consequently, it would be helpful to provide a server, a system, and a service provision method for providing a service to a user based on information related to a health checkup, to thereby raise incentives of the user to undergo a health checkup.

A server according to an embodiment of the present disclosure is for providing a service related to health of a user. The server includes a memory configured to store health checkup information of the user, and a controller configured to decide a service to be provided to the user based on the health checkup information, and to dispatch a vehicle loaded with equipment or goods for providing the service.

A system according to an embodiment of the present disclosure includes the server and a vehicle that provides the service. The server generates a dispatch schedule of the vehicle, and the vehicle is configured to be loaded with equipment or goods for providing the service, and driven based on the dispatch schedule.

Also, a method for providing a service related to health of a user according to an embodiment of the present disclosure includes acquiring health checkup information of the user, deciding a service to be provided to the user based on the health checkup information, and dispatching a vehicle loaded with equipment or goods for providing the service.

The server, the system, and the method disclosed herein provide a service to a user based on health checkup information, to thereby raise incentives of the user to undergo a health checkup.

### BRIEF DESCRIPTION OF THE DRAWINGS

In the accompanying drawings:

FIG. 1 is a diagram illustrating an exemplary configuration of a system according to an embodiment;

FIG. 2 is a diagram illustrating an exemplary configuration of a server according to an embodiment;

FIG. 3 is a flowchart illustrating an example of a service provision method by the server;

FIG. 4 is a conceptual diagram of a service to be provided to a user;

FIG. 5 is a diagram illustrating an exemplary configuration of a vehicle according to an embodiment; and

FIG. 6 is a sequence diagram illustrating an example of overall operations by a system according to an embodiment.

### DETAILED DESCRIPTION

Hereinafter, an embodiment for carrying out the present disclosure will be described with reference to the drawings. In the drawings, identical or similar components are denoted with the same signs.

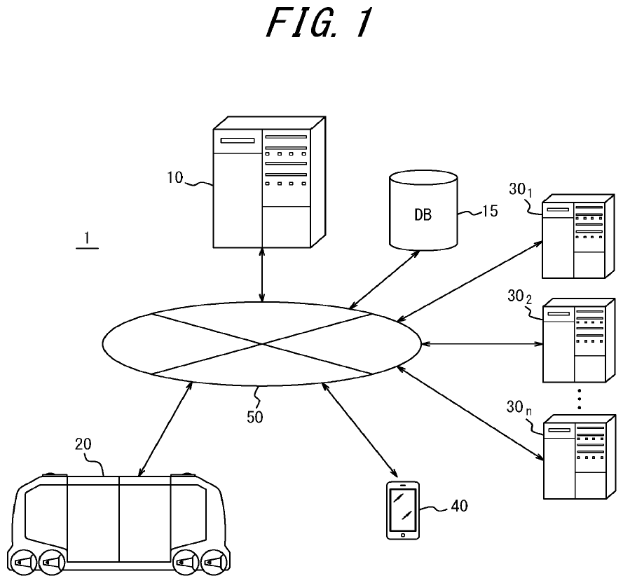

FIG. 1 is a diagram illustrating an exemplary configuration of a system that provides a service related to health of a user according to an embodiment of the present disclosure. A system 1 according to the present embodiment includes a server 10 and a vehicle 20. The server 10 and the vehicle 20 are connected by a network 50 of any type that allows for information communication. Additionally, in the system 1, a database (DB) 15, external servers 30 (30₁ to 30ₙ) of service providers, and a terminal apparatus 40 of a user are connected to the network 50 as necessary. Note that although one vehicle 20 and one terminal apparatus 40 are illustrated in FIG. 1, a plurality of each may also be connected to the network 50.

To give an overview of the present disclosure, the server 10 decides, based on health checkup information of a user, a service to be provided to the user, the service being related to health of the user (hereinafter, such service may also be referred to as ‘health-related service’). The vehicle 20 is configured to be loaded with equipment or goods for providing the health-related service. The server 10 dispatches the vehicle 20, and operates the vehicle 20 to provide the health-related service to the user based on a dispatch schedule. The equipment or goods are decided according to the service to be provided. The equipment or goods may include, for example, equipment for performing a health checkup, equipment for treating diseases, exercise equipment, rehabilitation equipment, massage equipment, health foods, supplements, and medicines.

The server 10 acquires health checkup information of the user from the database 15. Alternatively, the server 10 may acquire health checkup information of the user by some other means (for example, by receiving a health checkup result from the terminal apparatus 40). Herein, the term “user” refers to a user of the system 1, and includes both a person who has already undergone a health checkup and a person who has not yet undergone a health checkup. Also, the term “health checkup information of a user” refers to personal information such as the user's name, date of birth, and address (position information) and a health checkup result, and the information also includes information indicating that the user “has not yet undergone a health checkup”.

The server 10 decides a health-related service to be provided to the user based on the health checkup information of the user. The server 10 dispatches the vehicle 20 loaded with the equipment or goods for providing the service. Herein, the term dispatch is used to refer to: preparing the vehicle 20 loaded with the equipment or goods for providing the service; and driving (operating) the vehicle 20 based on a dispatch schedule. The configuration and operation of the server 10 will be described in detail later.

The database 15 stores information related to health checkups. For example, the database 15 may be owned by a government agency, and store information such as the names, addresses, and health checkup histories of citizens. The database 15 may be owned by a company subscribing to the system 1, and store health checkup histories and health checkup results of the employees and their families. The data base 15 may store health checkup results administered by a health institution.

The vehicle 20 provides service related to health of the user. Specifically, the vehicle 20 is loaded with equipment or goods for providing such health-related service and operated to go around a region where the user lives based on a dispatch schedule set by the server 10. The vehicle 20 may be a special-purpose vehicle provided with equipment and the like for a predetermined health-related service, or a general-purpose vehicle that can be loaded with equipment or goods for providing any of various types of health-related services. The system 1 may include a plurality of the vehicles 20. The types of equipment or goods to be loaded onto each vehicle 20 and the number of the vehicles 20 may desirably be arranged according to the service to be provided to the user. The equipment or goods for the service decided by the server 10 are loaded onto the vehicle 20, and thereafter, the vehicle 20 is driven based on a dispatch schedule to deliver the service to the user. As described later, the vehicle 20 may also perform autonomous driving or follow-up driving.

The external servers 30 (30₁ to 30ₙ) are owned by service providers subscribing to the system 1. The external servers 30 (30₁ to 30ₙ) receive health checkup information of a user from the server 10, analyze the health checkup information (a health checkup result, in particular), and transmit a proposal of a service that may be provided to the user, to the server 10. The service providers may be companies or institutions capable of providing various health-related services to users, such as a health institution, a pharmaceutical company, a food manufacturer, an exercise equipment manufacturer, or a sports gym. The service providers that own the external servers 30 provide the equipment or goods for the health-related service to be loaded onto the vehicle 20.

The terminal apparatus 40, which is a communication apparatus used by the user, may be a device such as a mobile phone, a smartphone, a smart speaker, or a personal computer (PC), without being limited thereto: the terminal apparatus 40 may be any type of apparatus used by the user.

The terminal apparatus 40 is capable of transmitting health checkup information of the user to the server 10. The terminal apparatus 40 may also receive information from the server 10. For example, the terminal apparatus 40 is capable of receiving information such as a dispatch schedule of the vehicle 20 that provides a health-related service and associated information on the health-related service. The dispatch schedule shows a schedule for dispatching the vehicle 20 that provides the service, and includes information such as locations to visit and times, a driving route, parking locations, and parking times. Also, the associated information on the health-related service is information associated with the service provided by the vehicle 20, and includes various information such as how to use the equipment or goods for providing the service, a product description, and usage guidelines.

The network 50 is a communication network of any type allowing the server 10, the vehicle 20, the external servers 30, and the terminal apparatus 40 to communicate with one another. The network 50 may be a wireless or wired transmission channel, and may be a communication network such as the Internet. For example, the network 50 in the present embodiment may include an ad hoc network, a metropolitan area network (MAN), a cellular network, a wireless personal area network (WPAN), the public switched telephone network (PSTN), a terrestrial wireless network, an optical network, some other network, or a combination of any of the above. A wireless network includes components such as an access point (for example, a Wi-Fi access point) and femtocells. Additionally, wireless communication equipment can be connected to a wireless network using Wi-Fi® (Wi-Fi is a registered trademark in Japan, other countries, or both), Bluetooth® (Bluetooth is a registered trademark in Japan, other countries, or both), cellular communication technology, or some other wireless technology and technology standard.

According to the system 1, the vehicle 20 loaded with equipment or goods for providing the health-related service is driven to go around a region where the user lives and deliver the service, thereby enabling the user to conveniently receive the health-related service. As a result, the system 1 contributes to: raising the user's incentives to undergo a health checkup increasing the ratio of users undergoing health checkups: and maintaining health of the users who underwent health checkups.

In the embodiment above, the database 15 is described as being connected to the network 50 of the system 1; however, the database 15 may be omitted in cases where the information stored in the database 15 is not used. The external servers 30 are described as being connected to the network 50: however, the external servers 30 may be omitted in cases where the system 1 does not use information from the external servers 30. If the transmission or reception of information with the terminal apparatus 40 is unnecessary, the terminal apparatus 40 may be omitted.

Next, the configuration and operation (service provision method) of the server 10 will be described in detail.

(Server Configuration)

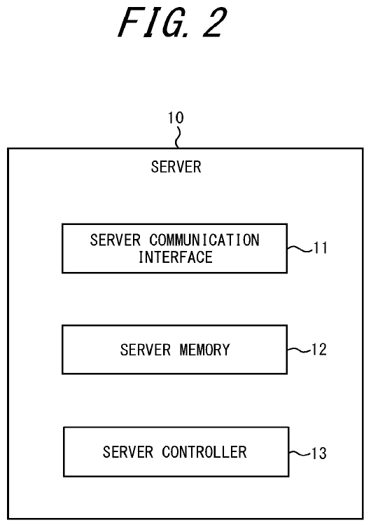

FIG. 2 is a diagram illustrating an exemplary configuration of the server 10 according to an embodiment. The server 10 includes a server communication interface 11, server memory 12, and a server controller 13.

The server communication interface (which may also be simplified as “communication interface”) 11 includes a communication module that connects to the network 50. The communication module supports mobile communication standards such as 4th Generation (4G) and 5th Generation (5G), a wired local area network (LAN) standard, or a wireless LAN standard for example, without being limited thereto. The communication module may support any type of communication standard. In the present embodiment, the server 10 is connected to the network 50 through the server communication interface 11. The server communication interface 11 receives (acquires) information (for example, health checkup information) transmitted from the database 15, the external servers 30, or the terminal apparatus 40 of the user over the network 50. The server communication interface 11 is also capable of transmitting information (such as, for example, a dispatch schedule) to the vehicle 20, the external servers 30, or the terminal apparatus 40 of the user over the network 50.

The server memory (which may also be simplified as memory) 12 includes semiconductor memory or magnetic memory. The server memory 12 may function as a main memory, an auxiliary memory, or a cache memory, for example. The server memory 12 stores information of any type to be used in the operation of the server 10. For example, the server memory 12 stores information such as a system program, an application program, and a management database. The information stored in the server memory 12 may be updated, for example, with information acquired from the network 50 through the server communication interface 11.

In the present embodiment, the server memory 12 stores health checkup information of a user acquired from at least one of the database 15 and the terminal apparatus 40 used by the user. The server memory 12 may store service to be provided to the user and a dispatch schedule of the vehicle 20 that provides the service. The server memory 12 may also store the operating status of the vehicle 20. The operating status of the vehicle 20 includes, for example, information related to the specifications of the vehicle 20, information on the equipment or goods loaded onto the vehicle 20.

The server controller (which may also be simplified as the controller) 13 includes one or more processors. In the present embodiment, the term “processor” refers to but is not limited to a general-purpose processor or a special-purpose processor specialized for a specific process. The server controller 13 controls the operation of the server 10 as a whole. For example, the server controller 13 controls the server 10 based on programs such as the system program and the application program stored in the server memory 12.

In the present embodiment, the server controller 13 decides a service to be provided to a user based on health checkup information of the user stored in the server memory 12. For example, in the case where the analysis of the health checkup result has identified the user's disease, the server controller 13 decides a service for treating the disease. Furthermore, in the case where the external servers 30 propose services to be provided to the user, the server controller 13 may decide which service is to be provided from among the proposed services. Information on the decided service is transmitted to the vehicle 20 and the external servers 30.

Next, the server controller 13 dispatches the vehicle 20 loaded with the equipment or goods to be used with the service, such that the service is appropriately provided to the user. The server controller 13 decides a dispatch schedule of the vehicle 20 and transmits the dispatch schedule to the vehicle 20, the external servers 30, and the terminal apparatus 40 through the server communication interface 11.

Also, the server controller 13 is capable of receiving driving information of the vehicle 20 (such as the position information and the traveling speed of the vehicle 20) from the vehicle 20 through the server communication interface 11. The server controller 13 stores the driving information in the server memory 12, and may correct the dispatch schedule of the vehicle 20 as necessary based on the stored information. The server controller 13 may also transmit update information for updating the dispatch schedule of the vehicle 20, the system program, the application program, or the like to the vehicle 20 through the server communication interface 11.

(Service Provision Method by Server)

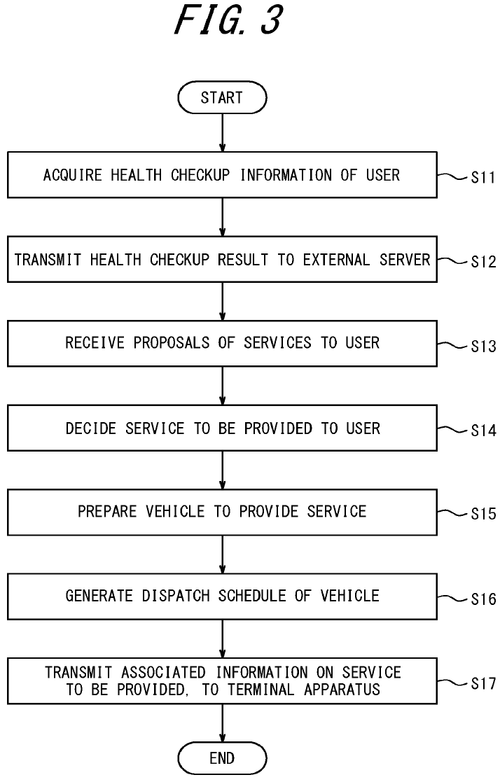

FIG. 3 is a flowchart illustrating an example of a service provision method by the server 10. The server 10 executes the following steps S11 to S17 to provide the health-related service to the user.

Step S11: The server 10 acquires health checkup information of the user from stored information in the database 15 or from the terminal apparatus 40 of the user. For example, the server 10 can connect to the database 15 storing user-related information over the network 50, and extract health checkup information of the user from the database 15. The server 10 may also receive information on a health checkup result for the user from the terminal apparatus 40 used by the user, and use the result data of a health checkup performed by the system 1. The server 10 stores the acquired health checkup information of the user in the server memory 12.

Step S12: The server 10 transmits the acquired health checkup information of the user to the external servers 30 (30₁ to 30ₙ). Note that the external servers 30 can analyze the received health checkup information of the user (a health checkup result in particular), and transmit proposals of services that may be provided to the user, to the server 10.

Step S13: The server 10 receives the proposals of services to the user transmitted from each of the external servers 30 (30₁ to 30ₙ).

Step S14: The server 10 decides the service to be provided to the user, where multiple types of services may also be provided. The server 10 may decide the service to be provided, from among the proposals of services to the user transmitted from the external servers 30. Additionally, the server 10 may also independently analyze the health checkup information of the user and decide the service to be provided to the user. In cases where the server 10 independently analyzes information on the user and decides the service to provide to the user, steps S12 and S13 may be omitted.

Step S15: The server 10 prepares the vehicle 20 to be loaded with equipment or goods for providing the service. As an example, the server 10 transmits information on the decided service to the vehicle 20 and the related external server(s) 30. In the case where a vehicle 20 already loaded with the equipment or goods for providing the service, the server 10 uses the vehicle 20 to provide the service to the user. Also, in the case where no vehicle is found to provide the service, the server 10 loads the vehicle 20 with the equipment or goods for providing the service. The vehicle 20 may be loaded in various ways. For example, the vehicle 20 may collect equipment or goods to be loaded based on the information on the decided service, and may automatically be loaded with such equipment or goods. Alternatively, the vehicle 20 may be driven to the service provider for the service (in many cases, the service provider that proposed the service), and receive the equipment or goods for providing the service from the service provider. At least part of the loading work may be performed manually.

Step S16: The server 10 generates a dispatch schedule of the vehicle 20 loaded with the equipment or goods for providing the service. Note that in order to deliver the service to the user efficiently, the dispatch schedule is generated based on the position information of the user (the address or the position information of the terminal apparatus 40). For example, in cases where a plurality of users are to receive the same service, the dispatch schedule is generated to go around and provide the service to the plurality of users. Thereafter, the dispatch schedule is transmitted to the vehicle 20 and the terminal apparatus 40.

Step S17: The server 10 additionally transmits associated information on the health-related service (such as information on the delivered equipment or goods) to the terminal apparatus 40 of the user, as necessary. In some cases, the associated information may be transmitted from each of the service providers.

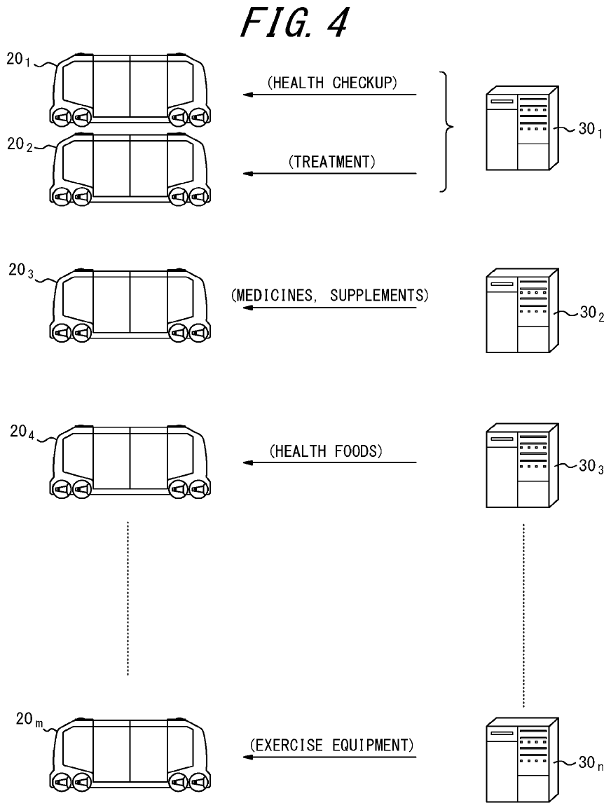

Next, a specific example of the health-related service to be provided to the user will be described. FIG. 4 is a conceptual diagram of the service to be provided to the user.

The external server 30₁ is a server managed by a medical institution that serves as a service provider, for example. The medical institution may analyze health checkup information of a user, and provide a service for undergoing a health checkup if the user has not undergone a health checkup over the past predetermined period. Specifically, examination equipment for performing a typical health checkup (such as a blood pressure gauge, blood testing equipment, an electrocardiograph machine, X-ray diagnostic imaging equipment, and an ultrasonographic device, for example) are loaded onto the vehicle 20, which is thus configured as a vehicle 20₁ to provide a health checkup service.

In the case where the medical institution analyzes a health checkup result of the user and finds that the user has a disease, the medical institution may provide a service for medical care. Specifically, treatment equipment to be used for treating the determined disease is loaded onto the vehicle 20 to configure a vehicle 20₂ that provides a disease treatment service. A physician or nurse may also ride in the vehicles 20₁ or 20₂ for providing the health checkup or disease treatment services. After the service is provided through the vehicle 20, the medical institution may also transmit, to the terminal apparatus 40 of the user, information such as advice as to when to have a health checkup in the future or as to the course of treatment, as associated information on the health-related service.

The external server 30₂ is managed by a pharmaceutical company that serves as a service provider, for example. In the case where the pharmaceutical company analyzes information related to a health checkup of a user (health checkup result) to find that the user has a disease, the pharmaceutical company may load medicine to be used to treat the disease thus found onto the vehicle 20, which is configured as a vehicle 20₃ that provides a pharmaceutical service. A pharmacist may also ride in the vehicle 20₃ for providing a pharmaceutical service. Alternatively, in order to manage the user's physical condition, the pharmaceutical company may load the vehicle 20 with supplements for improving one's physical condition, as as to have the vehicle 20 configured as a vehicle 20₃ that provides a supplement service. The supplements or the like may be free samples, and may be provided to the user free of charge. Furthermore, the pharmaceutical company may also transmit information such as instructions on how to use the provided medicine or supplements and information on other related medicines or supplements to the terminal apparatus 40 of the user, as associated information on the health-related service.

The external server 30₃ is managed by a food manufacturer that serves as a service provider, for example. In the case where the food manufacturer analyzes information related to a health checkup of a user (health checkup result) to find that the health of the user would be improved by food, the food manufacturer may load the vehicle 20 with health foods having a beneficial effect on health, so as to have the vehicle 20 configured as a vehicle 20₄ that provides a health food service. Note that the health foods may also be free samples, and may be provided to the user free of charge. Furthermore, the food manufacturer may also transmit information such as a product description of the provided health foods, cooking recipes using the health foods, and guidelines for improving one's diet using the health foods to the terminal apparatus 40 of the user as associated information on the health-related service.

Also, the external server 30ₙ is managed by an exercise equipment manufacturer or a sports gym that acts as a service provider, for example. In the case where the exercise equipment manufacturer or the sports gym analyzes information related to a health checkup of a user (health checkup result) to find that the health of the user would be improved by exercise, the exercise equipment manufacturer or the sports gym may load the vehicle 20 with exercise equipment having a beneficial effect on health, so as to have the vehicle 20 configured as a vehicle 20ₘ that provides an exercise equipment service. The exercise equipment is not limited to sports equipment, and may also be rehabilitation equipment or massage equipment. The vehicle 20ₘ that provides the exercise equipment service may be a mobile sports gym or a mobile sales vehicle for selling exercise equipment. A person such as an instructor, a trainer, or a caregiver may also ride in the vehicle 20ₘ. Furthermore, the exercise equipment company may also transmit information such as instructions on how to use the provided exercise equipment, training methods, and information on other related exercise gear to the terminal apparatus 40 of the user, as associated information on the health-related service.

Other than the above, a variety of service providers can analyze health checkup information of a user and propose health-related services. Also, a vehicle 20 loaded with equipment or goods for providing such a service can be dispatched to the user's location, so as to allow the user to receive the service at the user's home or in the vicinity thereof.

The server 10 is capable of deciding a service to be provided to the user from among the aforementioned services from the service providers, and operating the vehicles 20 (20₁ to 20ₘ) configured by the service providers based on a dispatch schedule. A plurality of services may also be provided to a user. For example, the vehicle 20₂ that provides the disease treatment service of the medical institution and the vehicle 20₃ that provides the pharmaceutical service of the pharmaceutical company can be dispatched to the same user at the same time.

(Vehicle Configuration)

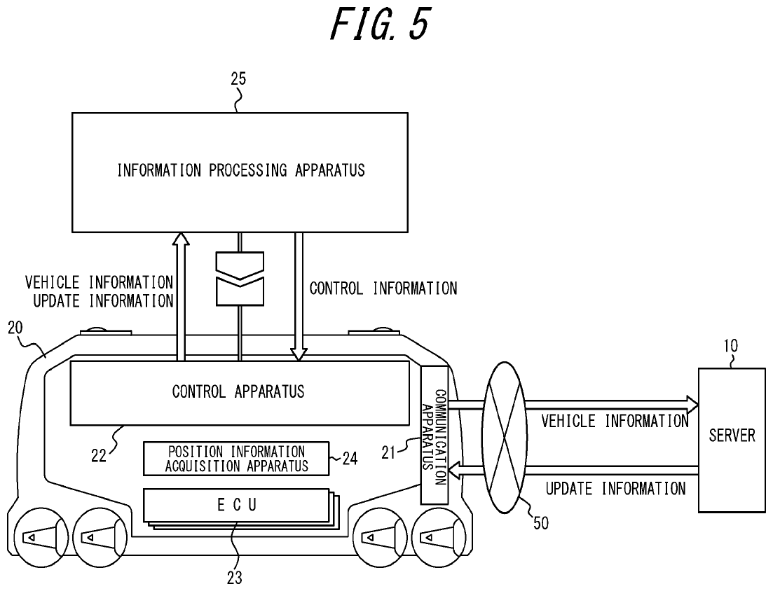

FIG. 5 is a diagram for explaining an exemplary configuration of the vehicle 20 according to an embodiment. The vehicle 20 includes, as auxiliary equipment, an information processing apparatus 25 that controls the vehicle 20. The vehicle 20, the information processing apparatus 25, and the server 10 are connected by the network 50 such as the Internet.

The vehicle 20 is an automobile capable of autonomous driving or follow-up driving, for example, but is not limited thereto. The vehicle 20 may be any type of vehicle that can be loaded with equipment or goods for providing a health-related service.

The vehicle 20 is controlled through cooperation by the vehicle 20 and the information processing apparatus 25. To summarize, the information processing apparatus 25 automatically generates control information according to autonomous driving control software, and transmits the generated control information to the vehicle 20. The vehicle 20 is controlled based on the received control information. The vehicle control is autonomous driving for example, but is not limited thereto. At least a part of an application programming interface (API) stipulating the specifications of the control information is disclosed to a service provider. The service provider is capable of using the disclosed API to freely program the autonomous driving control software of the information processing apparatus 25. Consequently, the service provider may load equipment according to the purpose onto the interior space of the vehicle 20 and program the autonomous driving control software using the API according to the purpose, to thereby provide a mobility service of any type.

In the present embodiment, the aforementioned equipment or goods for providing a health-related service are loaded onto the vehicle 20, and the vehicle 20 is driven to a destination by autonomous driving based on a dispatch schedule transmitted from the server 10.

The vehicle 20 includes a communication apparatus 21, a control apparatus 22, a plurality of electronic control units (ECUs) 23, and a position information acquisition apparatus 24. The apparatuses 21 to 24 are communicably connected via an on-board network such as a controller area network (CAN) or dedicated lines, for example.

The communication apparatus 21 may be an on-board communication module such as a data communication module (DCM), for example. The communication apparatus 21 may also include a communication module supporting mobile communications standard such as 4th Generation (4G) and 5th Generation (5G) to connect to the network 50.

The control apparatus 22 performs vehicle control based on the control information from the information processing apparatus 25. The vehicle control is autonomous driving to a destination for example, but is not limited thereto. Autonomous driving includes Levels 1 to 5 of driving automation defined by the Society of Automotive Engineers (SAE) for example, but is not limited thereto, and may be arbitrarily defined. Also, vehicle control is performed through cooperation by components such as the control apparatus 22 and each ECU 23. The control apparatus 22 includes a communication module that communicates with the information processing apparatus 25, the communication apparatus 21, and each ECU 23, one or more memories that store information such as a system program and an application program, and a controller including one or more processors that control the operation of the control apparatus 22 as a whole.

For example, the control apparatus 22 receives various kinds of vehicle information related to the vehicle 20 (such as speed, position, and autonomous driving status) from each of the ECUs 23. The control apparatus 22 transmits the vehicle information to the information processing apparatus 25, and also transmits the vehicle information to the server 10 through the communication apparatus 21. In addition, the control apparatus 22 receives update information for the system program and the like of the information processing apparatus 25 from the server 10 through the communication apparatus 21, and transmits the update information to the information processing apparatus 25. Also, in the case of acquiring control information from the information processing apparatus 25, the control apparatus 22 controls the vehicle 20 based on the control information.

The plurality of ECUs 23 cooperate with the control apparatus 22 to control the operation of the vehicle 20. Specifically, the plurality of ECUs 23 receive control instructions based on the control information, from the control apparatus 22, and control the operation of the vehicle 20 in accordance with the control instructions. For example, the plurality of ECUs 23 controls the operation quantities of the vehicle 20 so as to attain the values indicated by the control instructions. Additionally, at each control timing, the plurality of ECUs 23 collect measured values of control quantities or operation quantities of the vehicle 20 from various sensors installed in the vehicle 20, and transmit the measured values to the control apparatus 22.

The position information acquisition apparatus 24 includes one or more receivers that support an arbitrary satellite positioning system. For example, the position information acquisition apparatus 24 may include a Global Positioning System (GPS) receiver. The position information acquisition apparatus 24 acquires a measured value of the position of the vehicle 20 as position information, and transmits the position information to the control apparatus 22.

The information processing apparatus 25 includes a communication module that connects to the control apparatus 22 of the vehicle 20, one or more memories that store information such as a system program and an application program, one or more sensors that detect information related to the operation of the information processing apparatus 25 or the surrounding environment, and a controller including one or more processors that control the operation of the information processing apparatus 25 as a whole. The information processing apparatus 25 is installed in a position such as on the rooftop of the vehicle 20, for example. The position where the information processing apparatus 25 is installed is not limited to the above, and may also be installed at any position of the exterior or interior of the vehicle 20.

The information processing apparatus 25 receives vehicle information and update information from the control apparatus 22. The information processing apparatus 25 also generates control information based on information such as sensor information and vehicle information, and transmits the control information to the control apparatus 22. Additionally, the application program stored in the memory may include autonomous driving control software, and in this case, the information processing apparatus 25 functions as a autonomous driving kit according to the autonomous driving control software.

With this arrangement, the vehicle 20 is capable of performing autonomous driving or follow-up driving.

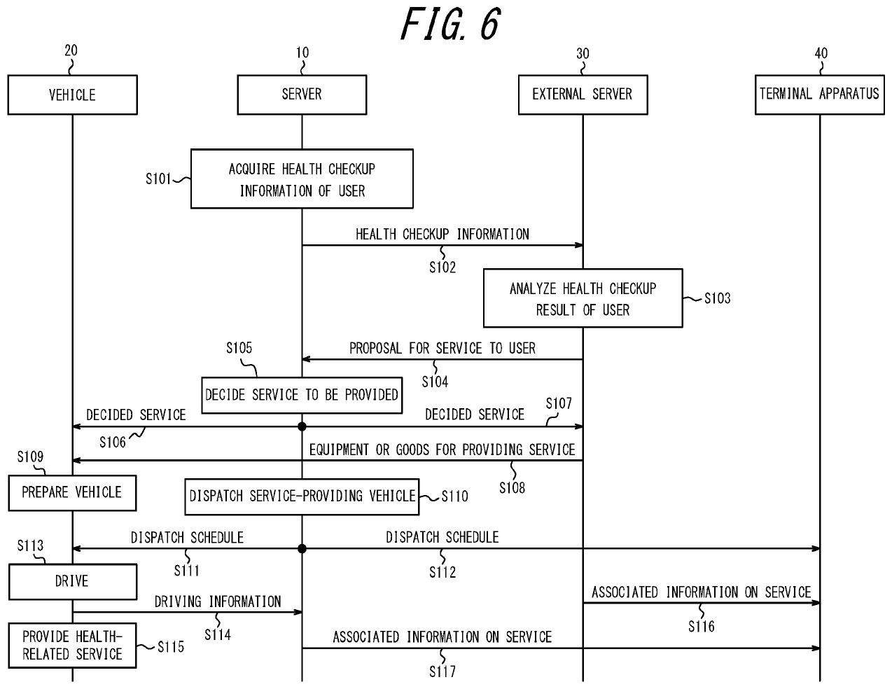

FIG. 6 is a sequence diagram illustrating an example of overall operations by the system 1 according to an embodiment. A procedure for providing a health-related service to a user will be described with reference to FIG. 6.

Step S101: The server 10 acquires health checkup information of a user. For example, the server 10 may acquire the health checkup information of a user from such source as the database 15, and may also receive a health checkup result for a user from the terminal apparatus 40 of the user.

Step S102: The server 10 transmits the health checkup information of the user (including the health checkup result) to the external server(s) 30.

Step S103: The external server(s) 30 analyzes the health checkup result for the user, and determines a service suited to the user.

Step S104: The external server(s) 30 transmits a proposal of a service that may be provided to the user, to the server 10.

Step S105: The server 10 decides the service to be provided to the user. The server 10 may also independently analyze the health checkup information of the user and decide the service to be provided to the user. Also, the server 10 may decide the service to be provided, from among the proposals of services to the user transmitted from the external server(s) 30.

Step S106: The server 10 transmits information on the decided service, to the vehicle 20. In the case where a vehicle 20 already loaded with the equipment or goods for providing the service exists, such vehicle 20 provides the service to the user.

Step S107: The server 10 transmits the information on the decided service, to the external server(s) 30. Note that the service provider who owns the external server(s) 30 possesses the equipment or goods for providing the service.

Step S108: In the case where no vehicle 20 corresponding to the service is found, the service provider who owns the external server(s) 30 supplies the equipment or goods for providing the service to the vehicle 20.

Step S109: The vehicle 20 to provide the service is prepared. In other words, the vehicle 20 is loaded with the equipment or goods for providing the service.

Step S110: The server 10 dispatches the vehicle 20 to provide the service. In other words, the server 10 prepares (selects) the vehicle 20 loaded with the equipment or goods for providing the service, and generates a dispatch schedule of the vehicle 20 based on the position information of the user.

Step S111: The server 10 transmits the dispatch schedule to the vehicle 20.

Step S112: The server 10 also transmits the dispatch schedule of the vehicle 20 to the terminal apparatus 40 of the user. In addition to transmitting the dispatch schedule, the server 10 may also transmit a push notification to the terminal apparatus 40 when the vehicle 20 approaches the user's location (home address or present position).

Step S113: The vehicle 20 starts driving based on the transmitted dispatch schedule.

Step S114: While traveling, the vehicle 20 transmits driving information, which includes information such as the position information and the traveling speed of the vehicle 20, to the server 10.

Step S115: The vehicle 20 uses the loaded equipment or goods for providing the service to provide a health-related service to the user.

Step S116: In the case where the service proposed by the external server(s) 30 is provided to the user, the external server(s) 30 can transmit associated information on the service provided by the vehicle 20 to the terminal apparatus 40.

Step S117: The server 10 can transmit the associated information on the service provided by the vehicle 20 to the terminal apparatus 40.

According to the present disclosure, the system 1 is capable of cooperating with the external servers 30 to provide a health-related service to a user using the vehicle 20.

A computer may suitably be used to function as the server 10 described above. In such a computer, a program stating processing content to achieve each function of the server 10 may be stored in a memory of the computer, and the functions can be implemented by reading and executing the program by a CPU of the computer. Note that the program may be recorded onto a computer-readable recording medium.

The foregoing embodiment is described as a representative example, and it should be obvious to persons skilled in the art that many alterations and substitutions are possible within the gist and scope of the present disclosure. Consequently, the present disclosure should not be interpreted as being limited by the foregoing embodiment, and various modifications or alterations are possible without departing from the scope of the claims. For example, it is possible to combine a plurality of function blocks described in the embodiment into a single block, or divide a single function block into a plurality of blocks.

1. A server for providing a service related to health of a user, comprising:
a memory configured to store health checkup information of the user; and
a controller configured to decide a service to be provided to the user based on the health checkup information, and to dispatch a vehicle loaded with equipment or goods for providing the service.

2. The server according to claim 1, wherein the health checkup information of the user is at least one of information stored in an external database and information on a health checkup result acquired from a terminal apparatus of the user.

3. The server according to claim 1, further comprising:
a communication interface configured to transmit the health checkup information to an external server, and also to receive, from the external server, a proposal of a service to be provided to the user, wherein
the controller decides a service to be provided to the user based on the received proposal of the service.

4. A system comprising:
the server according to claim 1; and
a vehicle configured to provide the service, wherein
the server generates a dispatch schedule of the vehicle, and
the vehicle is configured to be loaded with equipment or goods for providing the service, and driven based on the dispatch schedule.

5. The system according to claim 4, further comprising:
a terminal apparatus of the user, wherein
the terminal apparatus is configured to receive associated information on the service related to health of the user.

6. The system according to claim 4, wherein the vehicle performs autonomous driving or follow-up driving.

7. The system according to claim 4, wherein the vehicle is loaded with equipment to be used in a health checkup.

8. The system according to claim 4, wherein the vehicle is loaded with equipment to be used in disease treatment.

9. The system according to claim 4, wherein the vehicle is loaded with at least one of health foods, supplements, and medicines.

10. The system according to claim 4, wherein the vehicle is loaded with at least one of exercise equipment, rehabilitation equipment, and massage equipment.

11. A method of providing a service related to health of a user, comprising:
acquiring health checkup information of the user;
deciding a service to be provided to the user based on the health checkup information; and
dispatching a vehicle loaded with equipment or goods for providing the service.
